(19) United States
(12) Patent Application Publication
(10) Pub. No.: US 2011/0014094 A1
(43) Pub. Date: Jan. 20, 2011

(54) DISK TYPE MICROFLUIDIC DEVICE AND BLOOD TESTING APPARATUS USING THE SAME

(75) Inventors: Bong Gi KIM, Suwon-si (KR); Kab Tae HAN, Seongnam-si (KR); Walter KIM, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD., Suwon-si (KR)

(21) Appl. No.: 12/838,538

(22) Filed: Jul. 19, 2010

(30) Foreign Application Priority Data
Jul. 20, 2009 (KR) 10-2009-0065984
Jul. 9, 2010 (KR) 10-2010-0066241

(51) Int. Cl. G01N 35/00; B01L 3/00; B81B 7/00; G01N 33/48

(52) U.S. Cl. 422/400; 422/81; 422/506

(57) ABSTRACT

Disclosed herein are a disk type microfluidic device including a data area arranged thereon, and a blood testing apparatus having an information reading device for reading the data area of the disk type microfluidic device. The disk type microfluidic device includes a disk type body having a predetermined thickness, a plurality of chambers provided in the body to store a buffer or reaction solution, at least one channel to connect the plurality of chambers to one another, and a data area provided at a cylindrical portion of the body.

### CROSS-REFERENCE TO RELATED APPLICATION

This application claims the benefit of Korean Patent Application Nos. 10-2009-065984, filed on Jul. 20, 2009, and 10-2010-0066241, filed on Jul. 09, 2010 in the Korean Intellectual Property Office, the disclosure of which is incorporated herein by reference.

### BACKGROUND

1. Field

Embodiments of the present invention relate to a disk type microfluidic device, in which a plurality of microfluidic structures is arranged in a single disk type body, to enable implementation of various blood tests including, e.g., an immune serum test using blood and a biochemical test, and a blood testing apparatus to test blood using the disk type microfluidic device.

2. Description of the Related Art

A lab-on-a chip is an apparatus designed to perform tests including biochemical reactions on a small-sized chip, in which microfluidic structures are arranged on a chip type substrate to enable implementation of several steps of processing and operations.

Transferring a fluid in microfluidic structures may require a drive pressure, which may be a capillary pressure or a pressure produced by a separate pump. Recently, disk type microfluidic devices have been proposed, in which microfluidic structures are arranged in a disk type body and fluid moves in the microfluidic structures using centrifugal force to perform a series of operations. Such a disk type microfluidic device is referred to as a lab CD, lab-on a disk, or Digital Bio Disk (DBD).

In general, a disk type microfluidic device includes a chamber to hold a fluid therein, a channel for fluid flow, and a valve to control the fluid flow. The disk type microfluidic device may be made in various combinations of the above constituent elements.

To perform fluidic test using a disk type microfluidic device, a drive device (e.g., a blood testing apparatus) is generally used. The disk type microfluidic device may store various kinds of reagents to enable implementation of various kinds of tests. Conventional disk type microfluidic devices however do not store information related to, e.g., the kind and expiration date of the reagents.

When it is desired to store the above mentioned information in the disk type microfluidic device, a separate data area for information storage may be necessary, causing an increase in size of the disk type microfluidic device.

Moreover, providing the disk type microfluidic device with the separate data area may need to provide a blood testing apparatus with a device for identification of the data area. This may limit slimming of the blood testing apparatus.

### SUMMARY

Therefore, it is an aspect of an embodiment of the present invention to provide a disk type microfluidic device including a data area arranged on an exterior surface thereof.

It is another aspect of an embodiment of the present invention to provide a disk type microfluidic device with a data area to store a variety of information.

It is another aspect of an embodiment the present invention to provide a blood testing apparatus to perform a testing method (diagnosis operation), selected from a plurality of testing methods, based on information extracted from the data area of the disk type microfluidic device.

It is another aspect of an embodiment of the present invention to provide a blood testing apparatus to prevent an inaccurate test and reexamination of a disk type microfluidic device.

It is a further aspect of an embodiment of the present invention to provide a blood testing apparatus having an information reading device configured to read information from a data area included in a disk type microfluidic device. Additional aspects of embodiments of the invention will be set forth in part in the description which follows and, in part, will be obvious from the description, or may be learned by practice of the invention.

In accordance with an embodiment of the present invention, a disk type microfluidic device includes a disk type body having a predetermined thickness, a plurality of chambers provided in the body to store a buffer or reaction solution, at least one channel to connect the plurality of chambers to one another, and a data area provided at a cylindrical portion of the body.

The data area may store identification information of the disk type microfluidic device.

The identification information may include information related to whether or not the disk type microfluidic device is for a clinical chemistry test.

The identification information may include information related to the expiration date of the disk type microfluidic device.

The identification information may include the serial number of the disk type microfluidic device.

The data area may be in the form of a barcode.

The barcode may be of a two-dimensional barcode type.

The data area may include data stored in the form of two-dimensional matrix codes.

The body may be a resin injection molded product.

In accordance with an embodiment of the present invention, a blood testing apparatus includes a spindle motor to rotate a disk type microfluidic device in which a chamber, analysis site, and data area are formed, a valve opening device to selectively open a valve provided on a flow-path between the chamber and the analysis site, a blood testing device to read the analysis site, a data reading device to scan data area provided at a cylindrical portion of the disk type microfluidic device by irradiating light to the data area and to extract information included in the data area; and a controller to select a diagnosis operation from a plurality of diagnosis operations based on the extracted information and to control execution of the selected diagnosis operation.

The data reading device may include a barcode reader.

The controller may control to perform a diagnosis operation corresponding to the kind of the disk type microfluidic device.

The controller may determine whether or not the diagnosis operation will be performed, based on the expiration date of the disk type microfluidic device. The controller may determine whether or not a test is needed, based on the serial number of the disk type microfluidic device.

### BRIEF DESCRIPTION OF THE DRAWINGS

These and/or other aspects of the invention will become apparent and more readily appreciated from the following description of the embodiments, taken in conjunction with the accompanying drawings of which:

FIG. 1 is a plan view illustrating a configuration of a disk type microfluidic device according to an embodiment of the present invention;

FIG. 2 is a perspective view illustrating an external appearance of the disk type microfluidic device according to the embodiment;

FIG. 3 is a configuration view of a blood testing apparatus according to the embodiment; and

FIG. 4 is a perspective view illustrating an external appearance of a disk type microfluidic device according to another embodiment of the present invention.

### DETAILED DESCRIPTION

Reference will now be made in detail to the embodiments of the present invention, examples of which are illustrated in the accompanying drawings, wherein like reference numerals refer to like elements throughout.

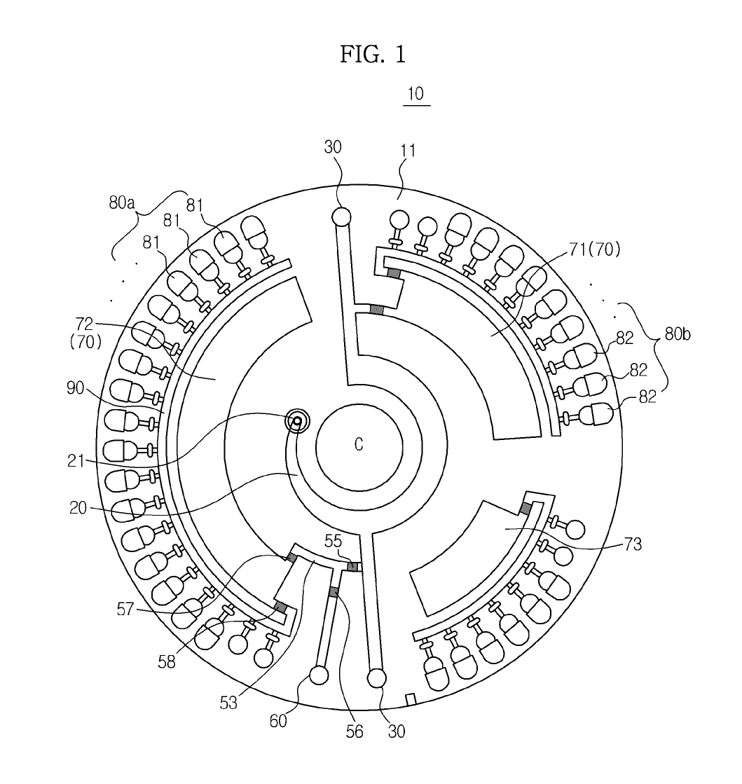

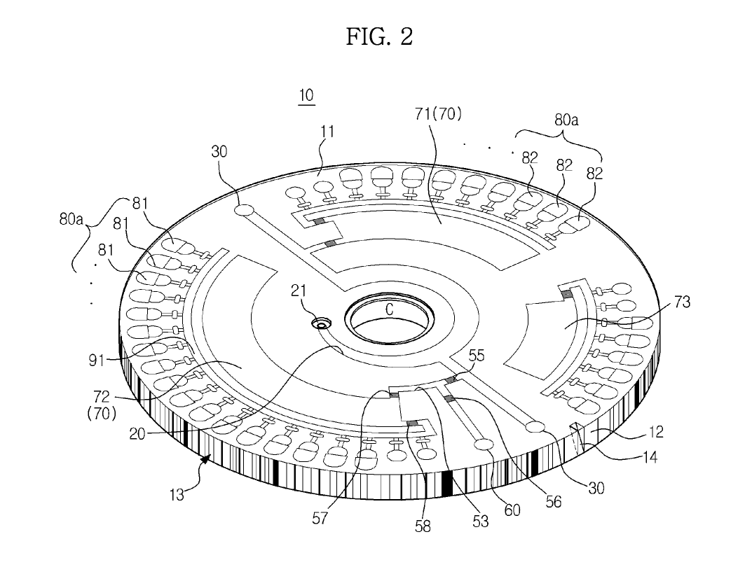

FIG. 1 is a view illustrating a disk type microfluidic device according to an embodiment of the present invention, and FIG. 2 is a perspective view of the disk type microfluidic device according to the embodiment.

As shown in FIGS. 1 and 2, the disk type microfluidic device 10 according to the present embodiment includes a rotatable body 11 (e.g., in the form of a disk), a plurality of fluid receiving chambers and a plurality of fluid moving channels defined in the body 11, and a data area 13 provided at a cylindrical portion 12 of the body 11.

The body 11 is rotatable about a center axis C thereof. In the chambers and channels defined in the body 11, e.g., the movement, centrifugal separation and mixing of a sample occurs under the influence of centrifugal force caused upon rotation of the body 11.

The body 11 may be made of plastics which have biologically inert superficial properties and are easy to mold, such as acryl, polydimethylsiloxane (PDMS), etc., but the embodiment is not limited thereto, and other materials having chemical and biological stability, optical transparency and mechanical processability may be utilized.

The body 11 may consist of multiple layers of plates. By providing interfaces of the contact plates with intagliated structures corresponding to, e.g., the chambers or channels and bonding the plates to each other, spaces and passages may be defined in the body 11. Bonding of the plates may be accomplished by various methods, such as adhesion using an adhesive or a double-sided tape, ultrasonic fusion, laser welding, etc.

Now, a series of structures arranged in the body 11 for a blood test will be described.

A sample chamber 20 is arranged in the body 11 at a radial inner position. The sample chamber 20 is defined to receive a predetermined amount of blood. A sample injection hole 21 is formed in an upper surface of the sample chamber 20, to inject blood into the sample chamber 20.

At least one serum separation chamber 30 is provided radially at the outside of the sample chamber 20, in which centrifugal separation of a sample occurs using rotation of the body 11.

In the serum separation chamber 30, heavy blood corpuscles settle downward by centrifugal force, whereas relatively light blood serums are located above the blood corpuscles thus being separated from blood.

A plurality of channels is provided at a side of the serum separation chamber 30. The plurality of channels includes a guide channel 53 to guide the serums separated in the serum separation chamber 30 to a dilution chamber 70 in which a dilution buffer is received.

The at least one dilution chamber 70 may include a plurality of dilution chambers to store different amounts of dilution buffers respectively as illustrated in the present embodiment. The plurality of dilution chambers 70 may have different volumes based on required volumes of dilution buffers. More particularly, two or more dilution chambers may be provided and in the present embodiment, first and second dilution chambers 71 and 72 to receive different volumes of dilution buffers are provided to change a dilution ratio.

A dilution chamber 73 may be provided, into which no sample is supplied from the serum separation chamber 30. The dilution chamber 73 is provided to obtain a standard value upon reaction detection and may store a dilution buffer. Reaction chamber groups 80a and 80b are arranged respectively at the outside of the corresponding first and second dilution chambers 71 and 72.

The first reaction chamber group 80a is provided at the outside of the corresponding first dilution chamber 71, and the second reaction chamber group 80b is provided at the outside of the corresponding second dilution chamber 72.

Each of the reaction chamber groups 80a or 80b includes one or more reaction chambers 81 or 82, and the reaction chambers 81 or 82 are connected to the corresponding dilution chamber 70 via a distribution channel 90 that distributes a dilution buffer. In a most simplified form, each reaction chamber group 80a or 80b may include a single reaction chamber.

The distribution channel 90, as shown in the drawings, may extend in a circumferential direction of the body 11, and may be connected to a corresponding dilution chamber 70 via a fourth valve 58 (that will be described hereinafter) interposed therebetween.

Reaction chambers 81 and 82 store reagents that cause different kinds of reactions with a sample (blood) respectively. In this case, the plurality of reaction chambers 81 or 82 included in the same reaction chamber group 80a or 80b may respectively store reagents suitable for reactions with a sample dilution buffer of the same dilution ratio.

For example, the first reaction chamber group 80a may store reagents including, e.g., triglycerides (TRIG), total cholesterol (CHOL), glucose (GLU), urea nitrogen (BUN), which react at a dilution ratio of serum to dilution buffer of 1:100. The second reaction chamber group 80b may store reagents including, e.g., direct bilirubin (DBIL), total bilirubin (TBIL), gamma glutamyl transferase (GGT), which react at a dilution ratio of serum to dilution buffer of 1:20.

That is, the sample dilution buffer, supplied from the second dilution chamber 72 into the plurality of reaction chambers 82 of the corresponding reaction chamber group 80b, has a different dilution ratio from that of the sample dilution buffer supplied from the first dilution chamber 71 into the plurality of reaction chambers 81 of the first reaction chamber group 80a. Therefore, the reaction chambers 81 or 82 of each reaction chamber group 80a or 80b may respectively store reagents suitable for the sample of the corresponding dilution ratio.

Although the reaction chambers 81 and 82 may have the same capacity, the embodiment of the present invention is not limited thereto, and the respective reaction chambers may have different capacities when different capacities of sample dilution buffers or reagents are necessary based on testing items.

Also, the plurality of reaction chambers 81 and 82 may have vents and injection holes, rather than taking the form of a hermetically sealed reaction chamber.

In the present embodiment, valves are provided at the channels connecting the respective chambers to one another.

The valves include a first valve 55 provided on a position of the guide channel 53 to open or close an exit of a serum separation chamber 30, a second valve 56 provided on a position of the guide channel 53 to open or close a serum removal chamber 60, a third valve 57 provided between the second valve 56 and the dilution chamber 70, and the fourth valve 58 provided at an exit of the dilution chamber 70 to open or close the distribution channel 90.

Although the respective valves may be any one of various kinds of valves including, e.g., capillary valves that are opened manually upon application of a predetermined pressure or more, and other valves that are operated actively upon receiving power or energy from an external source in response of operating signals, the present embodiment employs a phase transition valve that is operated via absorption of energy from an external source by way of example.

The valves are provided at the above described positions in a three-dimensional or planar arrangement between an upper plate and a lower plate of the body 11, thus serving to intercept fluid flow or to open the channels when they are displaced to an adjacent extra space via high temperature melting thereof.

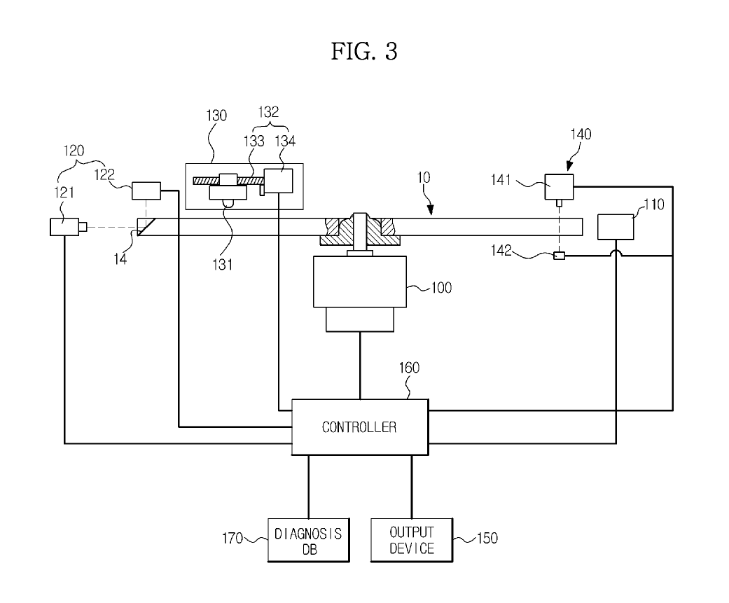

To apply heat to the valves 55, 56, 57 and 58, an external energy source (131, see FIG. 3) to emit light is movably arranged at the outside of the body 11 and may irradiate light to places where the valves 55, 56, 57 and 58 are located.

Accordingly, after the external energy source is moved, based on the test progress of the disk type microfluidic device, to the upper side of a valve that is necessary to be opened, the external energy source irradiates light downward, thereby opening the corresponding valve.

The respective valves may be made of a phase transition material and heating particles dispersed in the phase transition material.

Heating particles may have a size sufficient to freely move in channels having a width of several hundreds to thousands of micrometers. The heating particles are characterized in that the temperature of heating particles is increased rapidly to emit heat upon application of energy generated upon irradiation of light (e.g. laser). To obtain these properties, the heating particles may include a core containing a metal component and a hydrophobic shell. For example, the heating particles may include a core formed of Fe, and a shell surrounding the Fe core, the shell consisting of a plurality of surfactants. In commercially available products, the heating particles may be dispersed in carrier oil.

The phase transition material may be wax. When the heating particles transfer thermal energy converted from optical energy absorbed thereby, the wax is melted thus becoming fluid. Thereby, the valve is collapsed, opening the corresponding channel. The wax may have an appropriate melting point. An excessively high melting point increases a time required after irradiation of light begins and until the wax is melted, thus making it difficult to accurately control an opening time point. On the contrary, an excessively low melting point causes the wax to be partially melted without irradiation of light, resulting in unexpected fluid leakage. The wax may be paraffin wax, microcrystalline wax, synthetic wax, natural wax, or the like.

Alternatively, the phase transition material may be gel or thermoplastic resin. The gel may be polyacrylamide, polyacrylates, polymethacrylates, polyvinylamides, or the like. In addition, the thermoplastic resin may be cyclic olefin copolymer (COC), polymethylmethacrylate (PMMA), polycarbonate (PC), polystyrene (PS), polyoxymethylene (POM), perfluoralkoxy (PFA), polyvinylchloride (PVC), polypropylene (PP), polyethylene terephthalate (PET), polyetheretherketone (PEEK), polyamide (PA), polysulfone (PSU), polyvinylidene fluoride (PVDF), or the like.

In the disk type microfluidic device according to an embodiment, the cylindrical portion 12 is provided with a home position portion 14 in the form of a reflective member to set a reference position of the microfluidic device, and the data area 13 is provided at an outer surface of the cylindrical portion 12.

In an embodiment, the data area 13 is provided at the outer surface of the cylindrical portion 12 and therefore, may be realized using a dead space without increasing the size of the disk type microfluidic device.

In an embodiment, the data area 13 is used to store information in the form of a barcode.

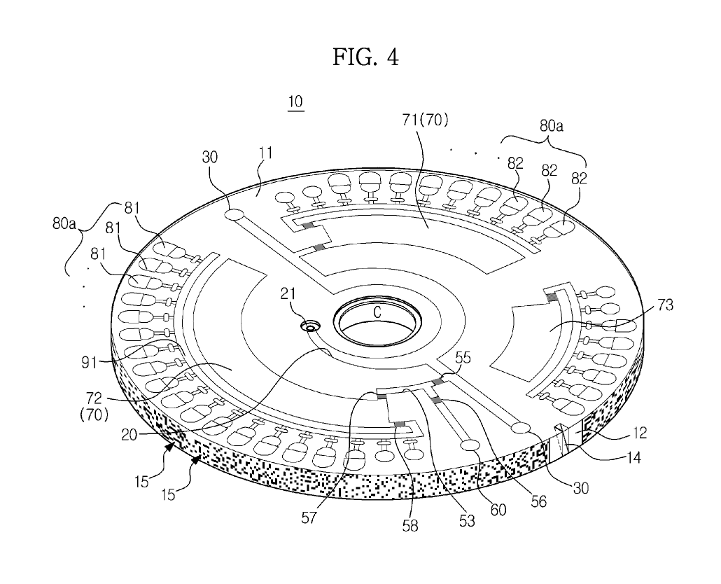

The barcode 13 may be a one-dimensional barcode as shown in FIG. 2, a two-dimensional barcode as shown in FIG. 4, or other various types of barcodes/matrix codes (e.g., two-dimensional matrix codes) to store a great quantity of information.

The barcode 13 may contain identification information of the disk type microfluidic device, such as information required to determine the kind of the disk type microfluidic device, information required to certify authenticity of the disk type microfluidic device, information related to the kind of a test, manufacturer information, information required to allow each disk type microfluidic device to perform a specific diagnosis operation, expiration date information, serial number, and the like. In addition to the above enumerated information, the barcode 13 of course may contain various other information if necessary.

Accordingly, in a blood testing apparatus, after the kind of the disk type microfluidic device 10 is determined by sensing the barcode 13 of the microfluidic device, a testing method suitable for the kind of the corresponding disk type microfluidic device is determined and the expiration date of the microfluidic device is confirmed prior to performing a test, to withhold the test if the microfluidic device has expired.

In addition, by sensing the barcode 13 of the disk type microfluidic device, it may be possible to confirm whether an object mounted in the blood testing apparatus is a disk type microfluidic device or not, and also, to certify whether the disk type microfluidic device is an authentic device or a counterfeit.

In addition, the serial number of the disk type microfluidic device 10 is confirmed, to prevent reexamination of the disk type microfluidic device if the corresponding microfluidic device has already been tested.

Next, the blood testing apparatus to test blood using the disk type microfluidic device according to the embodiment will be described.

FIG. 3 is a block diagram illustrating the blood testing apparatus according to the embodiment.

The blood testing apparatus according to the embodiment includes a spindle motor 100 to rotate the disk type microfluidic device, a data reading device 110, a home position setting device 120, a valve opening/closing device 130, a blood testing device 140, an output device 150, and a controller 160 to control the respective constituent elements.

The spindle motor 100 may rotate the disk type microfluidic device and more particularly, may stop or rotate the disk type microfluidic device to allow the reaction chambers 81 and 82 to reach specific positions.

Although not shown, the spindle motor 100 may include a motor drive mechanism to control an angular position of the disk type microfluidic device 10. For example, the motor drive mechanism may be one using a stepper motor or DC motor.

The data reading device 110 may be a barcode reader by way of example. To irradiate light to the data area 13 (e.g. a barcode) provided at the cylindrical portion 12 of the disk type microfluidic device 10 and receive the light reflected from the data area 13, the data reading device 110 is arranged parallel to the body 11 of the microfluidic device 10 so as to face the cylindrical portion 12 with a predetermined distance therebetween.

The data reading device 110 is arranged around the body 11 of the microfluidic device 10, rather than being arranged above or below the body 11, thus assuring slim configuration of the blood testing apparatus.

In the blood testing apparatus according to the present embodiment, it may be necessary to accurately set a reference point of the disk type microfluidic device for a high accuracy test.

For this, the blood testing apparatus may be provided with the home position setting device 120. The home position setting device 120 includes a light source 121, and an optical sensor 122 to generate electric signals upon receiving light from the light source 121.

The light source 121 is located at the outside of the cylindrical portion 12 at a height corresponding to a height of the microfluidic device 10, to irradiate light toward the cylindrical portion 12 of the microfluidic device 10. The optical sensor 122 is located above the microfluidic device 10, to receive light reflected from the microfluidic device 10.

Of course, e.g., a reflector may be provided on a position of an optical path that is used to set a home position, to enable setting of various optical paths.

The light from the light source 121 is reflected by the home position portion 14 and then, is introduced into the optical sensor 122. A light incident position on the optical sensor 122 is referred to as a home position.

The valve opening/closing device 130 is provided to open or close the valves 55, 56, 57 and 58 of the disk type microfluidic device 10. The valve opening/closing device 130 includes an external energy source 131, and a moving unit 132 to move the external energy source 131 to the valves 55, 56, 57 and 58 that need to be opened.

The external energy source 131 may be a laser light source to irradiate laser beams, or may be a light emitting diode lamp or a xenon lamp to irradiate visible light or infrared light. In particular, the laser light source may include at least one laser diode.

The moving unit 132 may include a drive motor 134, and a gear 133 to which the external energy source 131 is mounted, the gear 133 moving the external energy source 131 to the upper side of the valve to be opened, based on rotation of the drive motor 134.

The blood testing device 140 includes at least one light emitting element 141, and a light receiving element 142 arranged to correspond to the light emitting element 141, the light receiving element 142 serving to receive light having passed through the reaction chambers 81 and 82 of the microfluidic device 10.

The light emitting element 141 is a light source to be turned on and off at a predetermined frequency. Examples of available light sources include semiconductor light emitting devices, such as a Light Emitting Diode (LED), Laser Diode (LD), etc., and gas discharge lamps, such as a halogen lamp, a xenon lamp, etc.

The light emitting element 141 is positioned to allow light emitted therefrom to reach the light receiving element 142 through the reaction chambers 81 and 82.

The light receiving element 142 generates electric signals based on the intensity of incident light. For example, the light receiving element 142 may be a depletionlayer photo diode, avalanche photo diode (APD), photomultiplier tube (PMT), or the like.

Although the light emitting element 141 is arranged above the disk type microfluidic device 10 and the light receiving element 142 is arranged below the disk type microfluidic device 10 to correspond to the light emitting element 141 in the present embodiment, of course, positions of the light emitting element 141 and light receiving element 142 may be reversed. Also, although not shown, an optical path may be adjusted using a reflector, a light guide member, or the like.

The controller 160 controls the spindle motor 100, data reading device 110, home position setting device 120, valve opening/closing device 130, blood testing device 140, etc., to assure effective operations of the blood testing apparatus. Also, the controller 160 searches a diagnosis database (DB) 170 for comparative analysis between information detected from the blood testing device 140 and the diagnosis DB, thereby testing the presence of diseases of blood received in the reaction chambers 81 and 82 of the disk type microfluidic device 10.

The output device 150 serves to output the diagnosed results and the completion of operation. The output device 150 may be a visual output device, such as a Liquid Crystal Display (LCD), an audio output device, such as a speaker, or an audio-visual output device.

Next, of various operations of the blood testing apparatus according to the present embodiment, an operation related to the barcode reader will be described.

The barcode reader 110 transmits read results of data stored in the barcode 13 to the controller 160, and the controller 160 operates the respective constituent elements of the blood testing apparatus based on the read data.

Specifically, if the barcode reader 110 transmits sensed results of the barcode 13 of the disk type microfluidic device to the controller 160, the controller 160 determines the kind of the microfluidic device, thus determining a suitable testing method for the kind of the corresponding microfluidic device.

Based on the sensed results of the barcode 13 transmitted from the barcode reader 110 to the controller 160, the controller 160 may further confirm the expiration date of the microfluidic device. If the corresponding microfluidic device has expired, the controller 160 may withhold operations of the respective constituent elements of the blood testing apparatus and consequently, withhold a test.

Further, if the barcode reader 110 reads the serial number of the disk type microfluidic device, the controller 160 may determine, based on the read serial number transmitted from the barcode reader 110, whether or not the disk type microfluidic device has already been tested, thus preventing reexamination of the microfluidic device.

As is apparent from the above description, in a disk type microfluidic device according to the embodiment, a data area is provided in a cylindrical portion of a device body, resulting in slim size of the disk type microfluidic device.

Further, the data area stores identification information of the microfluidic device, such as, e.g., the kind, expiration date and serial number of the microfluidic device, thus allowing a blood testing apparatus to perform a suitable test for the corresponding microfluidic device.

Furthermore, providing a data reading device radially at the outside of the disk type microfluidic device may contribute to slimming of the blood testing apparatus.

While it is described in the above embodiment that an example of the data area includes a barcode, of course, the data area may take the form of a hologram, radio frequency identification (RFID) tag, or memory chip, used to store information therein.

In this case, the data reading device of the blood testing apparatus may take the form of a reader suitable to read information of the data area.

In addition, if the data area is a storage medium, such as e.g., a memory chip, to enable reading and writing of information, the data area may store information related to blood test results, patient information, blood collecting/testing date and time, and execution of a test, as well as identification information as described in the embodiment.

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A disk type microfluidic device comprising:
a disk type body having a predetermined thickness;
a plurality of chambers provided in the body to store a buffer or reaction solution;
at least one channel to connect the plurality of chambers to one another; and
a data area provided at a cylindrical portion of the body.

2. The device according to claim 1, wherein the data area stores identification information of the disk type microfluidic device.

3. The device according to claim 2, wherein the identification information includes information related to whether or not the disk type microfluidic device is for a clinical chemistry test.

4. The device according to claim 2, wherein the identification information includes information related to the expiration date of the disk type microfluidic device.

5. The device according to claim 2, wherein the identification information includes the serial number of the disk type microfluidic device.

6. The device according to claim 1, wherein the data area includes data in the form of a barcode.

7. The device according to claim 6, wherein the barcode is of a two-dimensional barcode type.

8. The device according to claim 1, wherein the data area includes data stored in the form of two-dimensional matrix codes.

9. The device according to claim 3, wherein the body is a resin injection molded product.

10. A blood testing apparatus comprising:
a spindle motor to rotate a disk type microfluidic device in which a chamber, analysis site, and data area are formed;
a valve opening device to selectively open a valve provided on a flow-path between the chamber and the analysis site;
a blood testing device to read the analysis site;
a data reading device to scan data area provided at a cylindrical portion of the disk type microfluidic device by irradiating light to the data area and to extract information included in the data area; and
a controller to select a diagnosis operation from a plurality of diagnosis operations based on the extracted information and to control execution of the selected diagnosis operation.

11. The apparatus according to claim 10, wherein the data reading device includes a barcode reader.

12. The apparatus according to claim 10, wherein the controller controls to perform a diagnosis operation corresponding to the kind of the disk type microfluidic device.

13. The apparatus according to claim 10, wherein the controller determines whether or not the diagnosis operation will be performed, based on the expiration date of the disk type microfluidic device.

14. The apparatus according to claim 10, wherein the controller determines whether or not a test is needed, based on the serial number of the disk type microfluidic device.
